(19) United States
(12) Patent Application Publication
(10) Pub. No.: US 2020/0101076 A1
(43) Pub. Date: Apr. 2, 2020

(54) PHARMACEUTICAL COMPOSITIONS FOR PREVENTION OR TREATMENT OF CYTOKINE RELEASE SYNDROME

(71) Applicant: Harrow Health, Inc., San Diego, CA (US)

(72) Inventors: Annette Marleau, San Diego, CA (US); Thomas Ichim, San Diego, CA (US); Dennis Elias Saadeh, Irvine, CA (US)

(73) Assignee: Harrow IP, LLC, San Diego, CA (US)

(21) Appl. No.: 16/582,316

(22) Filed: Sep. 25, 2019

Related U.S. Application Data
(60) Provisional application No. 62/738,145, filed on Sep. 28, 2018.

(51) Int. Cl. A61K 31/522; A61K 9/127

(52) U.S. Cl. CPC A61K 31/522; A61K 9/127

(57) ABSTRACT

Pharmaceutical compositions and methods for preventing, treating, or alleviating cytokine release syndrome are described, the compositions comprising pentoxifylline and a pharmaceutically acceptable carrier. Methods for using such compositions are also described.

### CROSS REFERENCE TO RELATED APPLICATION(S)

This application claims the benefit of priority under 35 U.S.C. § 119(e) of U.S. Ser. No. 62/738,145, filed Sep. 28, 2018, the entire content of which is incorporated herein by reference.

### FIELD OF THE INVENTION

The present invention relates generally to the field of pharmacology and more specifically to compositions and methods designed to treat, mitigate, and/or or prevent cytokine release syndrome, and to methods of preparing and using such compositions.

### BACKGROUND

The present disclosure relates to pharmaceutical formulations comprising xanthine or a xanthine derivative, such as pentoxifylline, and methods for treating cytokine release syndrome by local administration.

In the treatment of malignant tumors, neoplasms, and other cancer-related conditions, traditional methods that have been widely used include surgical treatments, radiological treatments, and chemotherapy. All these methods, while being useful in many situations, are characterized by many drawbacks and deficiencies, among which are toxic side effects, sometimes very severe ones, and limited ability to prevent metastasis and recurrence.

More recently, treatments based on the principles of immunology have emerged as promising kinds of treatments. Indeed, unlike more traditional methods mentioned above, immunotherapeutic treatments have been shown to be adequate in destroying at least some tumors while sparing healthy surrounding tissues and so are free of many deficiencies and side effects associated with earlier methods.

However, immunology-base treatments are not free of their own drawbacks and problems, one of which is so-called “cytokine release syndrome” which is the result of an exaggerated, massive production of cytokines in response to immunological interference. This effect may be dangerous and even life-threatening in some situations.

While there exist some methods and therapies designed to counteract cytokine release syndrome, none is problem-free. For instance, typical treatments of cytokine release syndrome that are commonly used include employing such compounds as corticosteroids and biological therapies (e.g., anti-IL6 therapies and anti-inflammatory agents). However, steroids may affect chimeric antigen receptor T-cells' activity and/or proliferation and put the patients in danger of sepsis and opportunistic infections. For example, dexamethasone administered 20 mg every 8 hours is one common practice. However, dexamethasone inhibits not only inflammation but also the ability of T-cells to be programmed for cytotoxicity. There are no readily available alternatives to steroids that can inhibit immunopathology without inhibiting active T-cell immunity.

Anti-inflammatory drugs which are also used to treat cytokine release syndrome may not be effective in controlling cytokine release syndromes or cytokine storms because the cytokine storm includes a very large number of cytokines while there is limited ability to infuse patients with anti-inflammatory drugs.

This specification discloses pharmaceutical compositions suitable for prevention, treatment, and/or alleviation of cytokine release syndrome that can achieve positive patient outcomes while being free of the above-mentioned and other drawbacks and deficiencies of existing formulations, and methods of fabricating and administering the same.

### SUMMARY

According to one embodiment of the invention, there is provided a method for preventing, treating, and/or alleviating cytokine release syndrome, in a mammalian subject in a need of the treatment. The method includes administering to the subject a pharmaceutical formulation comprising a therapeutically effective amount of a compound of formula I:

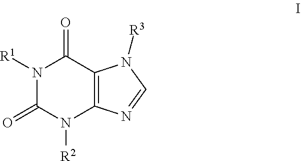

or a pharmaceutically acceptable salt, solvate or hydrate thereof, wherein each of R² and R³ is independently any of H, a C₁-C₆ alkyl, a C₂-C₆ alkenyl, a C₂-C₆ alkynyl, a cycloalkyl, a heterocyclyl, an aryl or a heteroaryl, each of which may be further optionally substituted.

According to another embodiment of the invention, the compound of formula I shown above is pentoxifylline.

### DETAILED DESCRIPTION

### A. Terms and Definitions

Unless specific definitions are provided, the nomenclatures utilized in connection with, and the laboratory procedures and techniques of analytical chemistry, synthetic organic and inorganic chemistry described herein, are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, formulating compositions and testing them. The foregoing techniques and procedures can be generally performed according to conventional methods well known in the art.

It is to be understood that both the foregoing general description and the following detailed description are exemplary and explanatory only and are not restrictive of the invention claimed. As used herein, the use of the singular includes the plural unless specifically stated otherwise. The section headings used herein are for organizational purposes only and are not to be construed as limiting the subject matter described.

As used herein, “or” means “and/or” unless stated otherwise. Furthermore, use of the term “including” as well as other forms, such as “includes,” and “included,” is not limiting.

“About” as used herein means that a number referred to as “about” comprises the recited number plus or minus 1-10% of that recited number. For example, “about” 100 degrees can mean 95-105 degrees or as few as 99-101 degrees depending on the context. Whenever it appears herein, a numerical range such as “1 to 20” refers to each integer in the given range; i.e., meaning only 1, only 2, only 3, etc., up to and including only 20.

The term “pharmaceutical composition” is defined as a chemical or biological compound or substance, or a mixture or combination of two or more such compounds or substances, intended for use in the medical diagnosis, cure, treatment, or prevention of disease or pathology.

The term “cytokine release syndrome” is defined as a kind of systemic inflammatory response syndrome (SIRS) arising as a result of one or several adverse events. Unusually severe cases of cytokine release syndrome known as “cytokine storm” are included in the definition of cytokine release syndrome for the purposes of this application. The following conditions or several adverse events causing SIRS are considered within the scope of cytokine release syndrome for the purposes of this application:
(a) caused by various diseases and injuries (arising as the body responds to them) such as, e.g., hemorrhage, pancreatitis, burns, ischemia, cardiac tamponade, anaphylaxis, adrenal insufficiency, complications of surgery, aortic aneurysmpulmonary embolism, or drug overdose;
(b) an adverse effect of some monoclonal antibody drugs, including anti-inflammatory, anti-cancer (such as, e.g., alemtuzumab, pembrolizumab, ranibizumab, ofatumumab, panitumumab, bevacizumab, betuximab, gemtuzumab ozogamicin, ipilimumab, rituximab, or trastuzumab), anti-viral, etc.;
(c) an adverse effect of various adoptive T-cell therapies.

For the purposes of the present invention, SIRS is further defined as being distinct and different from sepsis in that an active infection is found in sepsis. SIRS, on the other hand, for the purposes of the present invention, is defined as an inflammatory syndrome caused by non-infectious or traumatic causes in which patients exhibit at least two of the following four criteria:
1) body temperature that is either less than 36° C. or greater than 38° C.;
2) heart rate greater than 90 beats per minute;
3) tachypnea, with greater than 20 breaths per minute; or, an arterial partial pressure of carbon dioxide less than 4.3 kPa (32 mm Hg); and
4) white blood cell count less than 4,000 cells/mm³ (4×10⁹ cells/L) or greater than 12,000 cells/mm³ (12×10⁹ cells/L) or the presence of greater than 10% immature neutrophils (band forms).

The term “carrier” refers to a substance that serves as a vehicle for improving the efficiency of delivery and the effectiveness of a pharmaceutical composition.

The term “excipient” refers to a pharmacologically inactive substance that is formulated in combination with the pharmacologically active ingredient of pharmaceutical composition and is inclusive of bulking agents, fillers, diluents and products used for facilitating drug absorption or solubility or for other pharmacokinetic considerations.

The term “therapeutically effective amount” is defined as the amount of the compound or pharmaceutical composition that will elicit the biological or medical response of a tissue, system, animal or human that is being sought by the researcher, medical doctor or other clinician.

The term “pharmaceutically acceptable,” when used to defined a carrier, whether diluent or excipient, refers to a substance that is compatible with the other ingredients of the formulation and not deleterious to the recipient thereof.

The terms “administration of a composition” or “administering a composition” is defined to include an act of providing a compound of the invention or pharmaceutical composition to the subject in need of treatment.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance.

A “subject,” “individual,” or “patient,” is used interchangeably herein, which refers to a vertebrate, preferably a mammal, more preferably a human. Mammals include, but are not limited to, murines, simians, humans, farm animals, sport animals, and pets. Tissues, cells and their progeny of a biological entity obtained in vitro or cultured in vitro are also encompassed.

### B. Embodiments of the Invention

According to embodiments of the present invention, there is provided a method for preventing, treating, reducing, and/or alleviating cytokine release syndrome, in a mammalian subject in a need of the treatment. The method includes identifying the subject who is suffering or is likely to suffer in the near future from enhanced cytokine production followed by administering to such subject a pharmaceutically acceptable quantity of a pharmaceutical formulation comprising a therapeutically effective amount of at least one compound of formula I:

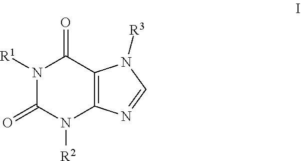

or a pharmaceutically acceptable salt thereof, wherein each of R¹, R² and R³ is independently selected from the group consisting of H, C₁-C₆ alkyl, C₂-C₆ alkenyl, C₂-C₆ alkynyl, cycloalkyl, heterocyclyl, aryl and heteroaryl, each of which is further optionally substituted.

A step of assessing impact of the pharmaceutical formulation on the enhanced cytokine production in the subject and adjusting the quantity thereof accordingly, if necessary, then follows. The composition may include a single compound of formula I or a combination of several such compounds, each of which is described by formula I.

The quantity of compound of formula I in the pharmaceutical formulation expressed as mass concentration can be between about 1.0 and about 75.0 mass % of compound of formula I, for example about 2.0 mass %. In some embodiments, the therapeutically effective amount of compound of formula I in the pharmaceutical formulation is between about 0.1 mg and about 20 mg such as between about 0.3 mg and about 10 mg, for example, about 0.5 mg. In one embodiment, the invention provides administration of a composition containing pentoxifylline in the quantity of about 20 mg/kg of the entire composition, intravenously, prior to administration of a cellular therapy possessing possibility of inducing immunoreactions. Initiation of therapy is performed ideally 4 hours prior to administration of the cellular therapy.

In some more particular embodiments, a compound of formula I may be administered prior to administration of a cellular therapy at the time of upregulation of biomarkers associated with cellular mediated immunopathology. In one such exemplary, non-limiting embodiment, a compound of formula I such as pentoxifylline may be administered, again, in the quantity of about 20 mg/kg to a patient who received a T-cell mediated therapy and who has at the same time a serum IFN-gamma higher than about 50 pg/ml and serum IP-10 higher than about 500 pg/ml. In another such exemplary, non-limiting embodiment, a compound of formula I such as pentoxifylline may be administered in a quantity of 20 mg/kg to a patient who received a T-cell mediated therapy and who has at the same time serum IL-6 concentrations higher than 200 pg/ml.

In one embodiment, the compound of formula I is a nonspecific phosphodiesterase inhibitor (PDEi) such as the above-mentioned pentoxifylline, i.e., 1-(5-oxohexyl)-3, 7-dimethylxanthine, i.e., a compound formula I where each of R² and R³ is methyl and R¹ is 5-oxohehyl, i.e., a functional group having the structure —(CH₂)₄—C(O)—CH₃. Lisofylline, an active metabolite of pentoxifylline, i.e., 1-(5-hydroxyhexyl)-3,7-dimethyl-3,7-dimethylxanthine can be also used if desired. The structure of lisofylline is basically the same as that of pentoxifylline except its functional group R¹ includes a primary alcohol moiety —C(OH)— instead of the acyl moiety —C(O)— that is present in the R¹ group in pentoxifylline. Other non-limiting examples of compounds encompassed by formula I that can be used include caffeine, aminophylline, enprofylline, isbufylline, theophylline, theobromine or 3-isobutyl-1-methylxanthine. In certain embodiments, the pharmaceutical formulation further comprises one or more additional active agent(s) that can be co-administered with a therapeutically effective amount of formula I. In one embodiment, compound of formula I such as pentoxifylline may be combined with activated protein C (activated drotrecogin alfa) known as XIGRIS® (Eli Lilly & Co.) at a dose of about 5-40 mcg/kg per hour, based on actual body weight for suppression of cytokine release syndrome. In another embodiment, the pharmaceutical formulation further comprises an additional active agent comprising FK506 (Tacrolimus) at a dose of about 0.2-0.5 mg/kg per day p.o. or 0.10-0.50 mg/kg per day given intravenously. In another embodiment, the pharmaceutical formulation further comprises one or more anticoagulants such as, but not limited to, heparin at 200-600 U/h, recombinant tissue factor pathway inhibitor (rTFPI) at 0.025-0.075 mg/kg per hour, rivaroxaban (XARELTO®) at 5-20 mg 1-2 times daily, Apixaben (ELIQUIS®) at 5-20 mg 1-2 times daily, Dabigatran (Pradaxa®) at 50-300 mg 1-2 times daily and/or warfarin at 5-10 mg/day.

In another embodiment, the pharmaceutical formulation also comprises an additional active agent and/or is co-administered with agents comprising anti-IL-6 monoclonal antibodies, anti-IL-6 receptor monoclonal antibodies, IL-6 receptor antagonist drugs, or other targeted inhibitor drug agents against IL-6 or IL-6 receptor. These agents may include but are not limited to the following monoclonal antibodies: siltuximab)(SYLVANT®), sirukumab, clazakizumab, olokizumab, or elsilimomab. In another embodiment, the antibodies against IL-6 receptor may comprise one or more of the following: tocilizumab (ACTEMRA®) and/or sarilumab) (KEVZARA®). In another embodiment, the compound of formula I such as pentoxifylline may be combined with antibodies that block TNF-alpha including but not limited to certolizumab pegol (CIMZIA), etanercept (ENBREL), adalimumab (HUMIRA), infliximab (REMICADE®), and golimumab)(SIMPONI®). In other embodiments, the pharmaceutical formulation comprises one or more additional active agents comprising anti-complement antibodies; specifically, therapeutic antibodies blocking the C₅a receptor, including but not limited to, eculizumab.

Without being bound to any theory, certain predominant events that are believed to lead to the progression of SIRS, and eventually to multiple organ failure, may be inhibited by a compound of formula I such as pentoxifylline according to embodiments of the invention. Such events are believed to include systemic activation of inflammatory responses, endothelial activation and initiation of the clotting cascade, associated with consumption of anticoagulants and fibrinolytic factors, complement activation, and organ failure and death. These pathological events appear to be related to each other, for example, it is known that complement activation stimulates the pro-coagulant state.

In cancer patients, SIRS may be initiated by several factors. For example, numerous patients receive immune suppressive chemo/radiotherapies that promote opportunistic infections. Additionally, given that many patients are cachectic, the low-grade inflammation causing the cachexia could augment the effects of additional bacterial/injury-induced inflammatory cascades. Finally, tumors themselves, and through interaction with host factors, have been demonstrated to generate systemically-acting inflammatory mediators such as IL-1, IL-6, and TNF-alpha that may predispose to SIRS. Again, without being bound to any theory, one potential mechanism of action of compound of formula I such as pentoxifylline is reduction of inflammatory cytokines such as TNF-alpha in order to suppression cachexia and enhance possibility of response to therapy.

In some embodiments, provided herein are methods for treating a severe manifestation of cytokine release syndrome, including its severe form, cytokine storm, where the cytokine release syndrome is associated with at least one cytokine such as TNF-alpha, IL-1 beta, IL-6, IL-33, CRP, IL-17, IL-2, IL-12, IL-18, HMGB-1, interferon gamma, and interferon alpha cytokines.

In some further embodiments, provided herein are methods for treating cytokine release syndrome associated with at least one medical condition such as a drop or reduction in blood pressure or a fever as well as when the cytokine release syndrome is caused by administration of at least one cancer immunotherapeutic, e.g., without limitation, chimeric antigen receptor (CAR) T-cells; or when the cytokine release syndrome is caused by at least one an infectious agent, e.g., without limitation, influenza, bird flu, severe acute respiratory syndrome (SARS), Epstein-Barr virus-associated hemophagocytic lymphohistiocytosis (HLH), bacterial sepsis, gram-negative sepsis, Dengue virus, malaria, Ebola virus, variola virus, or a systemic Gram-negative bacterial infection.

In some further embodiments, provided herein are methods for treating cytokine release syndrome associated with at least one non-infectious cause, such as, without limitation, hemophagocytic lymphohistiocytosis (HLH), sporadic HLH, macrophage activation syndrome (MAS), chronic arthritis, systemic Juvenile Idiopathic Arthritis (sJIA), Still's Disease, a Cryopyrin-associated Periodic Syndrome (CAPS), Familial Cold Auto-inflammatory Syndrome (FCAS), Familial Cold Urticaria (FCU), Muckle-Well Syndrome (MWS), Chronic Infantile Neurological Cutaneous and Articular (CINCA) Syndrome, a cryopyrinopathy comprising inherited or de novo gain of function mutations in the NLRP3 gene, a hereditary auto-inflammatory disorder, acute pancreatitis, severe burn injury, acute radiation syndrome, trauma, acute respiratory distress syndrome, systemic inflammatory response syndrome, and tumor lysis syndrome.

As stated above, cytokine release syndrome may arise as an immune reaction in response to certain T-cell therapies. Therefore, methods of counteracting such response according to embodiments of the present invention may be used in conjunction of many such therapies. Non-limiting examples of such T-cell therapies include CAR-T cell therapy where the chimeric antigen receptor (CAR) binds to an epitope of an antigen via an antibody or an antibody fragment that is directed to the antigen. In another embodiment, the antibody is a monoclonal antibody. Cytokine release syndrome may occur in infusion of antibody-based therapies including, but not limited to, anti-thymocyte globulin (ATG), the CD28 superagonist TGN1412, rituximab, obinutuzumab, alemtuzumab, brentuximab, dacetuzumab, and nivolumab. Cytokine release syndrome may also arise following administration of non-protein-based cancer drugs including, but not limited to, oxaliplatin and lenalidomide.

In another embodiment, the antibody is a polyclonal antibody. In another embodiment, the antibody fragment is a single-chain variable fragment (scFv). In another embodiment, the CAR T-cells bind to a tumor associated antigen (TAA). In some embodiments, the tumor associated antigen is any of the following: Mucin 1, cell surface associated (MUC1), polymorphic epithelial mucin, arginine-rich, mutated in early stage tumors (Armet), Heat Shock Protein 60 (HSP60), calnexin (CANX), methylenetetrahydrofolate dehydrogenase (NADP+ dependent) 2, methenyltetrahydrofolate cyclohydrolase (MTHFD2), fibroblast activation protein (FAP), matrix metallopeptidase (MMP6), B melanoma antigen-1 (BAGE-1), aberrant transcript of N-acetyl glucosaminyl transferase V (GnTV), Q5H943, carcinoembryonic antigen (CEA), Pmel, kallikrein-4, mammaglobin-1, MART-1, GPR143-OA1, prostate specific antigen (PSA), TRP1, tyrosinase, FGP-5, NEU proto-oncogene, Aft, MMP-2, prostate specific membrane antigen (PSMA), telomerase-associated protein-2, prostatic acid phosphatase (PAP), uroplakin II, or proteinase 3.

Other types of T-cell therapies where the methods of the present invention may be proved useful include those where a CAR binds to CD19 or CD20 to target B cells in the case where one would like to destroy B cells as in leukemia. In yet other embodiments, the CAR binds to any of ROR1, CD22, GD2, NY-ESO-1, MAGE family proteins, mesothelin, c-erbB2, mutational antigens that are tumor specific, such as BRAFV600E mutations and BCR-ABL translocations. In other embodiments, the CAR binds to any of viral antigens which are tumor-specific, such as EBV in HD, HPV in cervical cancer, and polyomavirus in Merkel cancer, Her2/neu antigen, α-folate receptor, or CAIX.

In yet other embodiments, the CAR binds to any of CD19, CD20, the CD22, CD23, CD24, CD30, CD33, CD38, CD44v6, CD44v7/8, CD123, CD171 antigens, or carcinoembryonic antigen (CEA). In another embodiment, the CAR binds to EGFRvIII. In other embodiments, the CAR binds to any of EGP-2, EGP-40, EphA2, Erb-B2, Erb-B 2, 3, 4, Erb-B3/4, FBP, fetal acetylcholine receptor, GD2, or GD3.

In other embodiments, the CAR binds to any of HER2, HMW-MAA, IL-11R alpha, IL-13R alphal, KDR, kappa-light chain, Lewis Y, L1-cell adhesion molecule, MAGE-A1, CMV infected cells, MUC1, MUC16, NKG2D ligands, NY-ESO-1 (amino acids 157-165), oncofetal antigen (h5T4), PSCA, PSMA, ROR1, TAG-72, VEGF-R2 or other VEGF receptors, B7-H6, CA9, αᵥβ₆ integrin, 8H9, NCAM, or fetal acetylcholine receptor.

In various embodiments, CAR T-cell both targets certain antigens and has therapeutic effect on subjects with certain diseases. For example, in some embodiments CAR T-cell targets the CD19 antigen and has a therapeutic effect on subjects with B-cell malignancies, ALL, follicular lymphoma, CLL, and lymphoma. Other exemplary, non-limiting embodiments of methods of the present invention where CAR T-cell both targets certain antigens and has therapeutic effect on subjects with certain diseases, are illustrated in Table 1.

TABLE 1
Exemplary Embodiments of CAR T-cell Targets and Effects

| No. | Target | Therapeutic Effect On |
|---|---|---|
| 1 | CD22 antigen | B-cell malignancies |
| 2 | Alpha folate receptor or | Ovarian cancer, epithelial |
| | folate receptor alpha | cancer |
| 3 | CAIX or G250/CAIX | Renal cell carcinoma |
| 4 | CD20 antigen | Lymphomas, B-cell |
| | | malignancies, B-cell |
| | | lymphomas, mantle cell |
| | | lymphoma, indolent B-cell |
| | | lymphomas |
| 5 | CD23 antigen | CLL |
| 6 | CD24 antigen | Pancreatic adenocarcinoma |
| 7 | CD30 antigen | Lymphomas, Hodgkin |
| | | lymphoma |
| 8 | CD33 antigen | AML |
| 9 | CD38 antigen | Non-Hodgkin lymphoma |
| 10 | CD44v6 antigen | Various malignancies |
| 11 | CD44v7/8 antigen | Cervical carcinoma |
| 12 | CD123 antigen | Myeloid malignancies |
| 13 | CEA antigen | Colorectal cancer |
| 14 | EGFRvII | Glioblastoma |
| 15 | EGP-2 | Multiple malignancies |
| 16 | EGP-40 | Colorectal cancer |
| 17 | EphA2 | Glioblastoma |
| 18 | Erb-B2 or ErbB3/4 | Various tumors including |
| | | breast cancer, prostate |
| | | cancer, and colon cancer |
| 19 | Erb-B 2, 3, 4 | Breast cancer, other |
| | | cancers |
| 20 | FBP | Ovarian cancer |
| 21 | Fetal acetylcholine | Rhabdomyosarcoma |
| | receptor | |
| 22 | G_{D2} | Neuroblastoma, melanoma, |
| | | or Ewing's sarcoma |
| 23 | G_{D3} | Melanoma |
| 24 | HER2 | Medulloblastoma, |
| | | pancreatic adenocarcinoma, |
| | | glioblastoma, osteosarcoma, |
| | | or ovarian cancer |
| 25 | HMW-MAA | Melanoma |
| 26 | IL-11Ralpha | Osteosarcoma |
| 27 | IL-13receptor alpha1 | Glioma, glioblastoma, |
| | | or medulloblastoma |
| 28 | IL-13 receptor alpha2 | Various malignancies |
| 29 | KDR | Tumor neovasculature |
| 30 | Kappa-light chain | B-cell malignancies |
| | | (B-NHL, CLL) |
| 31 | Lewis Y | Various carcinomas or |
| | | epithelial-derived tumors |
| 32 | L1-cell adhesion molecule | Neuroblastoma |
| 33 | MAGE-A1 or HLA-A1 MAGE A1 | Melanoma |
| 34 | Mesothelin | Mesothelioma |
| 35 | CMV infected cells | CMV |
| 36 | MUC1 | Breast or ovarian |
| | | cancer |
| 37 | MUC16 | Ovarian cancer |
| 38 | NKG2D ligands | Myeloma, ovarian, and |
| | | other tumors |
| 39 | NY-ESO-1 (157-165) or HLA- | Multiple myeloma |
| | A2 NY-ESO-1 | |
| 40 | Oncofetal antigen (h5T4) | Various tumors |
| 41 | PSCA | Prostate carcinoma |
| 42 | PSMA | Prostate cancer/tumor |
| | | vasculature |
| 43 | ROR1 | B-CLL and mantle cell |
| | | lymphoma |
| 44 | TAG-72 | Adenocarcinomas or |
| | | gastrointestinal cancers |
| 45 | VEGF-R2, other VEGF | Tumor neovasculature |
| | receptors | |
| 46 | CA9 | Renal cell carcinoma |
| 47 | CD171 antigen | Renal neuroblastoma |
| 48 | NCAM | Neuroblastoma |
| 49 | An angiogenic factor | Tumor vasculature |

Various angiogenic factors mentioned in Table 1 are considered within the scope of the instant invention, including, without limitation, VEGFR2, endoglin, angiogenin, angiopoietin-1, Del-1, acidic (aFGF) and basic (bFGF) fibroblast growth factors, follistatin, granulocyte colony-stimulating factor (G-CSF), hepatocyte growth factor (HGF)/scatter factor (SF), interleukin-8 (IL-8), leptin, midkine, placental growth factor, platelet-derived endothelial cell growth factor (PD-ECGF), Platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), vascular endothelial growth factor (VEGF)/vascular permeability factor (VPF), and an angiogenic protein which can be also a growth factor. I

In various embodiments, an angiogenic protein for use in the compositions and methods disclosed herein may be any of fibroblast growth factors (FGF), VEGF, VEGFR and neuropilin 1 (NRP-1), angiopoietin 1 (Angl) and Tie2, platelet-derived growth factor (PDGF; BB-homodimer) and PDGFR, transforming growth factor-beta (TGF-β), endoglin and TGF-β receptors, monocyte chemotactic protein-1 (MCP-1), integrins αV, β3, αV, β5 and α5β1, VE-cadherin and CD31, ephrin, plasminogen activators, plasminogen activator inhibitor-1, nitric oxide synthase (NOS), COX-2, AC133, Idl/Id3, an angiopoietin (e.g., angiopoietin 1, angiopoietin 3, angiopoietin 4 or angiopoietin 6). In one embodiment, endoglin is also known as CD105, EDG, HHT1, ORW, or ORW1. In one embodiment, endoglin is a TGF-β co-receptor. In another embodiment, the CAR T-cells bind to an antigen associated with an infectious agent such as Mycobacterium tuberculosis having associated antigens such as antigen 85B, lipoprotein IpqH, ATP dependent helicase putative, uncharacterized protein Rv0476/MTO4941 precursor, or uncharacterized protein Rv1334/MT1376 precursor.

In some embodiments, the pharmaceutical formulation further comprises a pharmaceutically acceptable excipient or carrier, including, but not limited to, an antioxidant, an adjuvant or synergist, and a preservative.

Non-limiting examples of antioxidants that can be used include α-tocopherol acetate, acetone sodium bisulfite, acetylcysteine, ascorbic acid, ascorbyl palmitate, butylated hydroxyanisole, butylated hydroxytoluene, cysteine, cysteine hydrochloride, d-a-tocopherol natural, d-a-tocopherol synthetic, dithiothreitol, monothioglycerol, nordihydroguaiaretic acid, propyl gallate, sodium bisulfite, sodium formaldehyde sulfoxylate, sodium metabisulfite, sodium sulfite, sodium thiosulfate, thiourea, tocopherols.

Non-limiting examples of the adjuvant or synergist include citric acid, EDTA (ethylenediaminetetraacetate) and salts, hydroxyquinoline sulfate, phosphoric acid, and tartaric acid.

Non-limiting examples of the preservatives are benzalkonium chloride, benzethonium chloride, benzoic acid and salts, benzyl alcohol, boric acid and salts, cetylpyridinium chloride, cetyltrimethyl ammonium bromide, chlorobutanol, chlorocresol, chorhexidine gluconate or chlorhexidine acetate, cresol, ethanol, imidazolidinyl urea, metacresol, methylparaben, nitromersol, o-phenyl phenol, parabens, phenol, phenylmercuric acetate/nitrate, propylparaben, sodium benzoate, sorbic acids and salts, β-phenylethyl alcohol, thimerosal. In particular embodiments, the preservative is benzyl alcohol.

As mentioned above, the pharmaceutical composition described herein may further optionally include one or several pharmaceutically acceptable excipient(s). Examples of suitable excipients include, but are not limited to, hyaluronic acid, hyalurnonidase or other agents capable of degrading hyaluronic acid. Hyaluronidase may be conjugated to a polymer or formulated or provided in a manner that increases the serum half-life thereof. The hyaluronidase can be administered together with pentoxifylline or administered separately. They can be administered sequentially or intermittently or simultaneously or in any order.

Additional non-limiting examples of carriers, excipients or diluents which may be included in the compositions of the present invention include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oils.

Polymers to be conjugated to hyalurnonidase prior to the administering the latter may include cellulose, pullulan, and polyalkyelene glycol. In some embodiments hyaluronidase can be modified by pegylation, which can be caused by reaction of hyaluronidase with the following exemplary non-limiting polymers (molecular weights shown are weight-averaged and also for illustration purposes only): methoxy-poly(ethylene glycol)-succinimidyl butanoate (mPEG-SBA) (5 kDa); methoxy-poly(ethylene glycol)-succinimidyl butanoate (mPEG-SBA) (20 kDa); methoxy-poly(ethylene glycol)-succinimidyl butanoate (mPEG-SBA) (30 kDa); methoxy-poly(ethylene glycol)-succinimidyl-α-methylbutanoate (mPEG-SMB) (20 kDa); methoxy-poly(ethylene glycol)-succinimidyl-α-methylbutanoate (mPEG-SMB) (30 kDa); methoxy-poly(ethylene glycol)-butyraldehyde (mPEG-butyraldehyde) (30 kDa), methoxy-poly(ethylene glycol)-succinimidyl propionate (mPEG-SPA) (20 kDa); methoxy-poly(ethylene glycol)-succinimidyl propionate (mPEG-SPA) (30 kDa); (methoxy-poly(ethylene glycol))₂-N-hydroxysuccinimide ester (mPEG₂-NHS) (10 kDa branched); (methoxy-poly(ethylene glycol))₂-N-hydroxysuccinimide ester (mPEG₂-NHS) (20 kDa branched); (methoxy-poly(ethylene glycol))₂-N-hydroxysuccinimide ester (mPEG₂-NHS) (40 kDa branched); (methoxy-poly(ethylene glycol))₂-N-hydroxysuccinimide ester (mPEG₂-NHS) (60 kDa branched); biotin-poly(ethylene glycol)-N-hydroxysuccinimide ester (biotin-PEG-NHS) (5 kDa biotinylated); poly(ethylene glycol)-p-nitrophenyl carbonate (PEG-p-nitrophenyl-carbonate) (30 kDa); or poly(ethylene glycol)-propionaldehyde (PEG-propionaldehyde) (30 kDa). In any of the examples, the polymer can be a PEG that has a molecular weight of 30 or about 30 kilodaltons.

In some embodiments, hyaluronidase may be administered in a dosage range amount of between about 0.01 μg/kg and about 100 μg/kg (body weight (BW) of the subject), such as between about 0.01 μg/kg and about 50 μg/kg, or between about 0.01 μg/kg and about 15 μg/kg, or between about 0.05 μg/kg and about 10 μg/kg, or between about 0.75 μg/kg and about 7.5 μg/kg, or between about 1.0 μg/kg and about 5.0 μg/kg, for example, between about 1.0 μg/kg and about 3.0 μg/kg. Expressed differently, the dosage may range between about 0.1 Unit/kg BW of the subject and about 5,000 Units/kg BW, such as between about 0.5 Unit/kg BW and about 4,000 Units/kg BW, or between about 1 Unit/kg BW and about 1,000 Units/kg, or between about 1 Unit/kg BW and about 500 Units/kg, or between about 5 Units/kg BW and about 500 Units/kg, or between about 10 Units/kg BW and about 500 Units/kg, for example, between about 20 Units/kg BW and about 400 Units/kg body BW.

In another embodiment, hyaluronidase can be formulated for sustained release, such as in lipid vesicles, including liposomes and other such vehicles. In some embodiments, hyaluronidase is administered at a concentration and frequency sufficient to enhance generation of hyaluronic acid cleavage products which active dendritic cells.

Other non-limiting examples of acceptable excipients that can be used include nonionic polyoxyethlene-polyoxypropylene block copolymers (e.g., of POLOXAMER® or PLURONIC® families (which can be used inter alia as surfactants as mentioned below), as well as poly(acrylic acid) in its various cross-linked or non-cross-linked versions, such as those belonging to the Carbomer 940® family of products. Other types of product that can be used in the excipient portion of the pharmaceutical formulation may be water-soluble methylcellulose and hydroxypropyl methylcellulose polymers, such as METHOCEL® family of products, for example, a hydroxypropyl methylcellulose product METHOCEL® E4M.

According to further embodiments, methods for fabricating the above-described pharmaceutical compositions are provided. A one-batch formulation method may be used, where the components of the pharmaceutical formulation can be combined in single container; the components may be added to the container simultaneously or consecutively. Alternatively, a two- or multiple-batch method(s) may be used if desired, where each component of the pharmaceutical formulation can be combined in separate container followed by combining the contents of each container.

Compositions of the present invention may be administered orally or parenterally, including intravenous, intra-arterial, intraperitoneal, intramuscular, intrasternal, topical, rectal, or intradermal route of administration. For use as an oral or parenteral formulation, pentoxifylline may be administered as a capsule, a tablet, a coated tablet, a slow-releasing tablet, granules, powder, syrup, a suspension, an emulsion, sap, an aerosol, and a suppository, and the parenteral preparation may be a sterilized aqueous solution, a non-aqueous solvent, a suspension, an emulsion, and a lyophilized preparation. Those having ordinary skill in the art will select the specific route of administration as well as the form in which the formulation is provided as to be most beneficial to a patient taking into account all the medical, pharmaceutical, and other relevant information and data.

More specifically, compositions of the present invention intended for oral administration may be formulated with pharmaceutically acceptable carriers which typically would include, without limitations, a diluent, a preservative, a binder, a lubricant, a disintegrant, a swelling agent, a filler, a stabilizer, and combinations thereof. Carriers may also include all the components of a coating composition which may include a plasticizer, a coloring matter, a colorant, a stabilizer, and a flow agent.

Non-limiting examples of suitable coating materials include cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers, acrylic acid copolymers, methacrylic resins, zein, shellac, and polysaccharides. Additionally, the coating materials may contain a typical carrier such as a plasticizer, a pigment, a colorant, a flow agent, a stabilizer, a pore former, and a surfactant. Optional pharmaceutically acceptable excipients include a diluent, a binder, a lubricant, a disintegrant, a colorant, a stabilizer, or a surfactant, but are not limited thereto.

Diluents are generally necessary to increase the volume of a solid dosage form, so that a particle size is provided for compression of tablets or formation of beads and granules. Non-limiting examples of suitable diluents include dicalcium phosphate dihydrate, calcium sulfate, lactose, sucrose, mannitol, sorbitol, cellulose, microcrystalline cellulose, kaolin, sodium chloride, dry starch, hydrolyzed starch, pregelatinized starch, silicone dioxide, titanium oxide, magnesium aluminum silicate, and powdered sugar.

Binders are used to impart cohesive properties to a solid dosage formulation, and thus ensure that a tablet or bead or granule remains intact even after the composition of the dosage forms. Non-limiting examples of suitable binder materials include starch, pregelatinized starch, gelatin, sugars (including sucrose, glucose, dextrose, lactose and sorbitol), polyethylene glycol, waxes, natural and synthetic gums such as acacia, tragacanth, and sodium alginate, cellulose including hydroxypropylmethylcellulose, hydroxypropylcellulose, ethyl cellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid, and polyvinylpyrrolidone.

One useful function of lubricants is to facilitate the process of fabrication of tablets. Non-limiting examples of suitable lubricants include magnesium stearate, calcium stearate, stearic acid, glycerol behenate, polyethylene glycol, talc, and mineral oil. Disintegrants are used to facilitate disintegration or breakup of the dosage form after administration, and generally include, without limitation, starch, sodium starch glycolate, sodium carboxymethyl starch, sodium carboxymethylcellulose, hydraxypropyl cellulose, pregelatinized starch, clays, cellulose, alginine, gums or cross-linked polymers, such as cross-linked PVP. Stabilizers are used to inhibit or retard drug decomposition reactions which include, for example, oxidative reactions. Non-limiting examples of suitable stabilizers include antioxidants, butylated hydroxytoluene (BHT), ascorbic acid, and salts and esters thereof; vitamin E, tocopherol and salts thereof, sulfites such as sodium metabisulphite, cysteine and derivatives thereof, citric acid, propyl gallate, and butylated hydroxyanisole (BHA).

In some embodiments, oral dosage formulations, such as capsules, tablets, solutions, and suspensions, may be formulated for controlled release. For example, one or more compounds and optional one or more additional active components may be formulated into nanoparticles, microparticles, and combinations thereof, and encapsulated (including nanoencapsulation) in a soft or hard gelatin or non-gelatin capsule or dispersed in a dispersing medium to form an oral suspension or syrup. The particles may be formed of the drug and a controlled release polymer or matrix. Alternatively, the drug particles may be coated with one or more controlled release coating agents prior to incorporation into a finished dosage form.

In the practice of the invention, it may be required to administer a high initial dose of pentoxifylline at initiation of therapy, in order to generate a high plasma concentration. This may be achieved through parenteral administration of the compound. The preparation for parenteral administration may be prepared as an aqueous composition using technologies known to those having ordinary skill in the art. Generally, such compositions may be prepared as injectable formulations, for example, solutions or suspensions; solid forms such as micro or nanoparticles, suitable for use to prepare solutions or suspensions upon the addition of a reconstitution medium prior to injection; emulsions, such as water-in-oil (w/o) emulsions or oil-in-water (o/w) emulsions, and microemulsions thereof, liposomes, or emulsomes.

The carrier may be a solvent or dispersion medium containing, for example, water, ethanol, one or more polyols (for example, glycerol, propylene glycol, and liquid polyethylene glycol), oils (for example, vegetable oils (peanut oil, corn oil, sesame oil, and the like)), and combinations thereof, but is not limited thereto. The suitable fluidity may be maintained by using a coating material, such as lecithin, by maintaining the required particle size in the case of dispersion, or by using a surfactant. In addition, it is possible to include an isotonic agent sugars or salts (for example, sodium chloride), but the isotonic agent is not limited thereto.

Solutions or dispersions of the active compounds as a free acid, a free base or pharmaceutically acceptable salts may be prepared in water or another solvent or dispersing medium suitably mixed with one or more pharmaceutically acceptable excipients. Non-limiting examples of suitable excipients include surfactants, dispersants, emulsifiers, pH modifying agents, and combinations thereof. Non-limiting examples of suitable surfactants include anionic, cationic, amphoteric or nonionic surface-active agents. Non-limiting examples of suitable anionic surfactants include those containing carboxylate, sulfonate, and sulfate ions inclusive of sodium, potassium, and ammonium of long chain alkyl sulfonates and alkyl aryl sulfonates such as sodium dodecylbenzene sulfonate, dialkyl sodium sulfosuccinates, such as sodium dodecylbenzene sulfonate; dialkyl sodium sulfosuccinates, such as sodium bis-(2-ethylthioxyl)-sulfosuccinate, and alkyl sulfates such as sodium lauryl sulfate.

Non-limiting examples of suitable cationic surfactants include quaternary ammonium compounds such as benzalkonium chloride, benzethonium chloride, cetrimonium bromide, stearyl dimethylbenzyl ammonium chloride, polyoxyethylene and coconut amine. Non-limiting examples of suitable of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, products of POLOXAMER® or PLURONIC® family, stearoyl monoisopropanolamide, and polyoxyethylene hydrogenated tallow amide. Examples of amphoteric surfactants include sodium N-dodecyl-alanine, sodium N-lauryl-iminodipropionate, myristoamphoacetate, lauryl betaine, and lauryl sulfobetaine.

It will be understood by those having ordinary skill in the art that the specific dose level and frequency of dosage for any particular patient may be varied and will depend upon a variety of factors including the activity of the specific compound employed, the metabolic stability and length of action of that compound, the age, body weight, general health, gender, diet, and the severity of the particular ophthalmological condition being treated.

In additional embodiments, pharmaceutical kits are provided. The kit includes a sealed container approved for the storage of pharmaceutical compositions, the container containing one of the above-described pharmaceutical compositions and a device for administering the formulation. An instruction for the use of the composition and the information about the composition are to be affixed to the container or otherwise enclosed with it.

The following examples are provided to further elucidate the advantages and features of the present invention, but are not intended to limit the scope of the invention. The examples are for the illustrative purposes only. USP pharmaceutical grade products were used in preparing the formulations described below.

### Example 1. Using Pentoxifylline as a Premedication for Preventing Infusion Reactions to the Monoclonal Antibody, Rituximab

Candidates for therapy according to this example include B cell prolymphocytic leukemia, chronic lymphocytic leukemia, and non-Hodgkin's lymphoma. Patients who may be treated with the pharmaceutical formulation are those deemed to be at-risk for cytokine release syndrome including patients considered to have high tumor burdens or high baseline lymphocyte counts exceeding 50.0×10⁹/L.

Patients may be pre-treated with pentoxifylline at a dose of 20 mg/kg formulated for intravenous delivery and administered on the same day, and prior to, the infusion of rituximab. In the hours following the pentoxifylline pretreatment and retuximab infusion, the subsequent occurrence of cytokine release syndrome in pentoxifylline-pretreated patients may be monitored on the basis of two or more of the following clinical symptoms of fever, chills, nausea, vomiting, hypotension, 5 to 10-fold increases in liver enzymes, elevation of D-dimer (for blood clotting), elevation in levels of IL-6 in plasma or serum, and/or prolongation of prothrombin time. Effective dosages of this formulation may be adjusted thereafter based on the condition of the patient with respect to the abovementioned parameters.

### Example 2. Using Pentoxifylline for Treatment of Grade 1 Cytokine Release Syndrome after CAR T Therapy

A patient may be first treated with fludarabine and cyclophosphamide for lymphodepletion on days −4 through −2, and then be given CD19 CAR T cells on day 0. The patient may as a result develop fever on day 1 that peaks at about 40.7° C. associated with rigors, which may persist for three days. Additionally, an IL-6 serum level of >500 pg/mL may be measured at that time where an IL-6 concentration of >200 pg/mL may be classified as prognostic of cytokine release syndrome.

The pharmaceutical formulation consisting of pentoxifylline formulated as an oral suspension at a concentration of 10 mg/kg may be administered to the patient twice daily. Stopping of treatment with the pentoxifylline formulation is indicated by 24 hours without fever or rigors. Stopping of treatment also requires that the patient not have development or persistence of flu-like symptoms and is hemodynamic stable based on measurements that include heart rate and blood pressure.

### Example 3. Using of Pentoxifylline for Treatment of Grade 3 Cytokine Release Syndrome after CAR T Cell Therapy for Leukemia

A patient who has received CAR T cell therapy is admitted to the hospital with symptoms of fever, headache, tachycardia, hypotension, and requiring supplemental oxygen. The patient may be treated with a pharmaceutical formulation of pentoxifylline at 20 mg/kg administered intravenously. Treatment with the pharmaceutical formulation may continue until supplemental oxygen is no longer required and the patient meets discharge criteria for the hospital. Thereafter, treatment with an oral suspension of pentoxifylline at 10 mg/kg may be prescribed for an additional two weeks.

### Example 4. Using Pentoxifylline for Treatment of a Patient with Posttransplantation Lymphoproliferative Disorder

A patient with Hodgkin's lymphoma has received a 9/10 HLA DRB1 mismatched unrelated hematopoietic stem cell transplant following conditioning with total body irradiation, fludarabine, and alemtuzumab. This patient may be treated with the pharmaceutical formulation upon presenting with rapidly progressive EBV-positive post-transplant lymphoproliferative disease (EBV-PTLD) 3 months after the transplant with elevated EBV DNA (>100,000 copies/10⁶ cells) and extensive nodal involvement on positron emission tomography. The pharmaceutical formulation of pentoxifylline may be administered to the patient at a dose of 20 mg/kg intravenously daily until resolution of EBV-PTLD as confirmed by a biopsy confirming an absence of histopathological evidence of lymphoproliferation.

### Example 5. Evaluating Efficacy of Pentoxifylline for Treatment of Severe Cytokine Release Syndrome Induced by CAR T Cell Therapy

A clinical study may be performed on patients who have received CAR T cell therapies for hematological malignancies and who have been subsequently diagnosed with a first episode of cytokine release syndrome. Inclusion criteria include patients who require advanced supportive care, which is a criterion for severe or life-threatening cytokine release syndrome. Treatment arms may consist of the following: A) tocilizumab; B) tocilizumab and pentoxifylline (administered together or separately). Tocilizumab may be administered intravenously at 4-12 mg/kg daily, or otherwise according to standard of care practices. A therapeutic formulation comprising pentoxifylline may be administered intravenously concurrent or on the same day as tocilizumab treatments. The therapeutic formulation of pentoxifylline may be administered as a bolus injection or continuously via the intravenous route. Normalization of hemodynamics (i.e., stabilization of blood pressure) and decreasing oxygen requirements allow the treatment to be discontinued. Recrudescence of cytokine release syndrome, if any, will be noted and will necessitate reinstatement of the therapy.

An analysis of the requirement for additional medications (e.g., glucocorticoids, other immunosuppressive agents) may be included as one of the study endpoints. The timing of resolution of cytokine release syndrome is defined as the patient having a lack of fever and being off vasopressors for at least 24 hours. A study endpoint will be the mean time (in days) of resolution of cytokine release syndrome, compared between Treatment Arms A and B. Patient responses to therapy may be defined by one or more criteria; for example: (a) cytokine release syndrome that is resolved within 7 days of the first dose of treatment with Treatment Arms A or B; and, (b) if no more than two doses of Treatment Arms A or B are required; and/or, (c) if no drugs other than those in Treatment Arms A or B plus corticosteroids are used. The two Treatment Arms may be compared in terms of the percentages of patient responders.

### Example 6. Guidance for Determining the Formulations and Dosing and of Pentoxifylline for Treatment of Cytokine Release Syndrome

The Penn grading scale for cytokine release syndrome, using clinical features, can be used as guidance for determining the formulations and dosing of the pharmaceutical formulation of pentoxifylline that will be administered to patients. This example guidance may be applied to treatment of patients that have undergone T cell therapies or other indications associated with cytokine release syndrome.

| | | | Administration |
|---|---|---|---|
| | | Pharmaceutical | Dose/Route of |
| Penn Grading | Clinical Symptoms to Indicate Treatment | Formulation of | Pharmaceutical |
| Scale | with Pharmaceutical Formulation | Pentoxyfylline | Formulation |
| Grade 1 | Mild reaction; patient needs supportive | Pentoxifylline | 5-10 mg/kg |
| | care such as antipyretics and antiemetics. | suspension | daily/Oral |
| Grade 2 | Moderate reaction; hospitalization for | Pentoxifylline | 5-20 mg/kg |
| | symptoms including fever, neutropenia, | suspension | daily/Oral or5-20 |
| | need for IV therapies (excluding fluid | | mg/kg injected |
| | resuscitation). | | intramuscularly |
| Grade 3 | More severe reaction; hospitalization, | Pentoxifylline | 20-40 mg/kg |
| | hypotension treated with intravenous | plus | daily/Intravenous |
| | fluids (defined as multiple fluid boluses | hyaluronidase | |
| | for blood pressure support) or low-dose | | |
| | vasopressors, coagulopathy requiring | | |
| | fresh frozen plasma or cryoprecipitate or | | |
| | fibrinogen concentrate, and hypoxia | | |
| | requiring supplemental oxygen. | | |
| Grade 4 | Life threatening; hypotension needing | Pentoxifylline | 20-60 mg/kg |
| | high-dose vasopressors and/or hypoxia | plus | daily/intravenous |
| | needing mechanical ventilation. | hyaluronidase | |

### Example 7. Preparing a Pharmaceutical Formulation

A pharmaceutical formulation intended for injections may be prepared as described above. The following components may be used in the quantities indicated below:
(a) about 2.0 g of pentoxifylline powder;
(b) about 15.0 g of lyophilized salt-free hyaluronidase;
(c) about 0.25 g of granulated sodium chloride;
(d) about 0.1 g of disodium edetate powder;
(e) about 0.059 g of calcium chloride dihydrate powder;
(f) about 0.188 g of monobasic anhydrous sodium phosphate;
(g) a quantity of 1% sodium hydroxide aqueous solution for adjusting pH; and
(h) about 100.0 mL of sterile for injection water.

All the dry components except pentoxifylline may be mixed together and dissolved in a quantity of water maintaining the pH between about 7.2 and 7.4 using NaOH, followed by adding pentoxifylline and the balance of water again keeping the pH at the same level. The final product may then be filtered through a 0.2 micron filter into pre-sterilized amber vials, stopped, sealed and crimped.

Although the invention has been described with reference to the above examples, it will be understood that modifications and variations are encompassed within the spirit and scope of the invention. Accordingly, the invention is limited only by the following claims.

What is claimed is:

1. A method for preventing, treating, reducing, or alleviating cytokine release syndrome in a mammalian subject in need thereof, comprising:
(a) identifying a mammalian subject who is suffering, or is likely to suffer in the near future, from an enhanced cytokine production;
(b) administering to the subject a pharmaceutically acceptable quantity of a pharmaceutical formulation comprising a therapeutically effective amount of at least one compound of formula I: 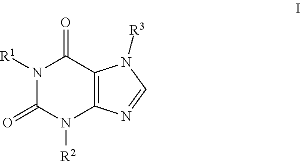
or a pharmaceutically acceptable salt thereof, wherein:
each of R¹, R² and R³ is independently selected from the group consisting of H, C₁-C₆ alkyl, C₂-C₆ alkenyl, C₂-C₆ alkynyl, cycloalkyl, heterocyclyl, aryl and heteroaryl, each of which is further optionally substituted; and
(c) assessing the impact of the pharmaceutical formulation on the enhanced cytokine production and adjusting the quantity thereof accordingly,
wherein said administering of the pharmaceutical formulation is conducted prior to, or after, the occurrence of the enhanced cytokine production,
thereby preventing, treating, or alleviating the cytokine release syndrome in the subject.

2. The method of claim 1, wherein the enhanced cytokine production requiring administering of the pharmaceutical formulation is detected by the patient exhibiting at least one condition selected from the group consisting of:
1) a body temperature that is less than about 36° C. or greater than about 38° C.;
2) a heart rate greater than about 90 beats per minute;
3) tachypnea, with greater than about 20 breaths per minute or an arterial partial pressure of carbon dioxide less than about 4.3 kPa; and
4) a white blood cell count less than about 4,000 cells/mm³ or greater than about 12,000 cells/mm³, or the presence of greater than about 10% immature neutrophils (band forms).

3. The method of claim 1, wherein the compound of formula I is a nonspecific phosphodiesterase inhibitor.

4. The method of claim 3, wherein the nonspecific phosphodiesterase inhibitor is selected from the group consisting of pentoxifylline, caffeine, aminophylline, enprofylline, isbufylline, theophylline, theobromine and 3-isobutyl-1-methylxanthine.

5. The method of claim 4, wherein the nonspecific phosphodiesterase inhibitor is pentoxifylline.

6. The method of claim 1, wherein the cytokine release syndrome is associated with at least one cytokine selected from a group consisting of TNF-alpha, IL-1 beta, IL-6, IL-33, CRP, IL-17, IL-2, IL12, IL-18, HMGB-1, interferon gamma, and interferon alpha cytokines.

7. The method of claim 1, wherein the cytokine release syndrome is associated with at least one medical condition selected from the group consisting of reduction in blood pressure and a fever.

8. The method of claim 1, wherein the cytokine release syndrome is caused by administration of at least one cancer immunotherapeutic.

9. The method of claim 8, wherein the cancer immunotherapeutic comprises chimeric antigen receptor (CAR) T cells.

10. The method of claim 1, wherein the cytokine release syndrome is caused by at least one infectious agent.

11. The method of claim 10, wherein the infectious agent is selected from a group consisting of influenza, bird flu, severe acute respiratory syndrome (SARS), Epstein-Barr virus-associated hemophagocytic lymphohistiocytosis (HLH), bacterial sepsis, gram-negative sepsis, Dengue virus, malaria, Ebola virus, variola virus, and a systemic Gram-negative bacterial infection.

12. The method of claim 1, wherein the cytokine release syndrome is associated with at least one non-infectious cause.

13. The method of claim 12, wherein the non-infectious causes are selected from a group consisting of hemophagocytic lymphohistiocytosis (HLH), sporadic HLH, macrophage activation syndrome (MAS), chronic arthritis, systemic Juvenile Idiopathic Arthritis (sJIA), Still's Disease, a Cryopyrin-associated Periodic Syndrome (CAPS), Familial Cold Auto-inflammatory Syndrome (FCAS), Familial Cold Urticaria (FCU), Muckle-Well Syndrome (MWS), Chronic Infantile Neurological Cutaneous and Articular (CINCA) Syndrome, a cryopyrinopathy comprising inherited or de novo gain of function mutations in the NLRP3 gene, a hereditary auto-inflammatory disorder, acute pancreatitis, severe burn injury, acute radiation syndrome, trauma, acute respiratory distress syndrome, and systemic inflammatory response syndrome.

14. The method of claim 1, wherein the pharmaceutical formulation is a liposomal formulation.

15. The method of claim 1, wherein the pharmaceutical formulation is a nanoencapsulated formulation.

16. The method of claim 15, wherein the liposomal formulation is targeted towards macrophages in the subject.

17. The method of claim 1, wherein the route of administration of the pharmaceutical formulation is selected from the group consisting of oral, intravenous, intra-arterial, intraperitoneal, intramuscular, intrasternal, topical, rectal, and intradermal route of administration.

18. The method of claim 1, wherein the pharmaceutical formulation further comprises at least one pharmaceutically acceptable excipient, carrier, antioxidant, adjuvant, synergist, or preservative.

19. The method of claim 18, wherein the excipient is hyaluronic acid or hyalurnonidase.

20. A kit comprising:
(a) a pharmaceutical formulation comprising a therapeutic effective amount of at least one compound of formula I: 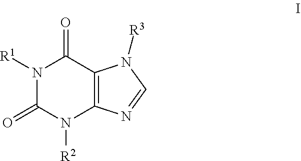
or a pharmaceutically acceptable salt thereof, wherein:
each of R¹, R² and R³ is independently selected from the group consisting of H, C₁-C₆ alkyl, C₂-C₆ alkenyl, C₂-C₆ alkynyl, cycloalkyl, heterocyclyl, aryl and heteroaryl, each of which is further optionally substituted;
(b) a device for administering the formulation;
(c) a container for housing the formulation and the delivery device; and
(d) a label and instructions for use affixed to, or enclosed with, the container.
